(19) United States
(12) Patent Application Publication
(10) Pub. No.: US 2015/0059453 A1
(43) Pub. Date: Mar. 5, 2015

(54) FUEL PROPERTY DETECTION APPARATUS FOR CONSTRUCTION MACHINE

(71) Applicant: KOBELCO CONSTRUCTION MACHINERY CO., LTD., Hiroshima-shi (JP)

(72) Inventors: Bocheng LI, Hiroshima (JP); Tomotaka KITA, Hiroshima (JP)

(73) Assignee: KOBELCO CONSTRUCTION MACHINERY CO., LTD., Hiroshima-shi (JP)

(21) Appl. No.: 14/449,356

(22) Filed: Aug. 1, 2014

(30) Foreign Application Priority Data
Aug. 27, 2013 (JP) 2013-175419

(51) Int. Cl. G01N 33/22; F02M 37/00

(52) U.S. Cl. CPC G01N 33/22; F02M 37/0088
USPC 73/64.56

(57) ABSTRACT

Provided is an apparatus for detecting property of fuel in a construction machine, with use of a sub-tank, the apparatus being capable of minimizing an influence on the detection given by water contained in fuel. The apparatus includes a sub-tank between an engine and a fuel tank of the construction machine, a fuel introducing pipe through which fuel is introduced to the sub-tank from the fuel tank, a fuel discharger through which fuel in the sub-tank is discharged to the engine, and a sensor. The sub-tank has a bottom section having a bottom fuel outlet for discharging fuel from the bottom section. The fuel discharger includes a bottom fuel discharging pipe connected to the bottom fuel outlet.

### BACKGROUND OF INVENTION

1. Technical field

The present invention relates to a fuel property detection apparatus for detecting a property of fuel supplied to an engine in a hydraulic excavator or other construction machines in order to determine the adequacy of the fuel.

2. Background art

There have been known apparatuses that each are adapted for detecting a property of fuel supplied to an engine provided in a hydraulic excavator or other construction machines in order to prevent a breakage of the engine or generation of inferior exhaust gas due to an inadequacy of fuel used in the engine. The apparatus is adapted to detect, for example, a physical quantity such as kinematic viscosity and density, or a chemical property.

Japanese Unexamined Patent Publication No. 2011-94549 (Patent Document 1) discloses a so-called sub-tank type apparatus. This apparatus includes a small sub-tank disposed in a fuel supply line connecting an engine with a fuel tank, and a sensor disposed on a bottom section of the sub-tank. The sensor detects a property of the fuel under a condition of no flow velocity and a stabilized flow rate of the fuel in the sub-tank. Also, Japanese Unexamined Patent Publication No. 2008-261812 (Patent Document 2) discloses a technique of detecting a fuel property inside a fuel tank.

The sub-tank type apparatus disclosed in Patent Document 1 can be easily mounted in an existing construction machine in an additional manner as compared to the apparatus disclosed in Patent Document 2, and is further capable of accurate and stable detection of fuel property; however, there is a likelihood that the sensor comes into contact with water contained in the fuel (or water separated from the fuel) to thereby decrease detection accuracy. Furthermore, water has the greater specific gravity than fuel (for example, light oil), and is likely to accumulate on the bottom of the sub-tank, thus significantly decreasing the detection accuracy of the sensor particularly in an apparatus where the sensor is disposed on the bottom of the fuel tank, thus disabling the apparatus from detecting fuel property Besides, even a sensor disposed on a location other than the bottom of the fuel tank cannot be completely prevented from contact with water, because the water having accumulated on the bottom of the fuel tank can be moved in the sub-tank by the flow of fuel or stirring currents accompanied by this flow.

### SUMMARY OF THE INVENTION

An object of the present invention is to provide an apparatus for detecting a property of fuel used in a construction machine including an engine and a fuel tank, the apparatus being capable of detecting a property of fuel securely and with high accuracy, with use of a sub-tank.

Provided by the present invention is an apparatus provided on a construction machine including an engine-and a fuel tank for containing fuel to be supplied to the engine to detect a property of fuel, the apparatus comprising: a sub-tank disposed between the engine and the fuel tank to contain fuel to be supplied from the fuel tank to the engine; a fuel introducing pipe provided between the sub-tank and the fuel tank to allow fuel to be introduced from the fuel tank to the sub-tank therethrough; a fuel discharger provided between the sub-tank and the engine to allow fuel to be discharged to the engine from the sub-tank therethrough; and a sensor provided inside the sub-tank to generate a detection signal concerning a property of the fuel in the sub-tank. The sub-tank has a bottom section having a bottom fuel outlet for discharging fuel through the bottom section. The fuel discharger includes a bottom fuel discharging pipe connected to the bottom fuel outlet.

### BRIEF DESCRIPTION OF DRAWINGS

FIG. 1 is a schematic diagram illustrating a system including a fuel property detection apparatus according to an embodiment of the present invention.

FIG. 2 is a cross sectional view illustrating a configuration of a sub-tank of the apparatus and flows of fuel.

FIG. 3 is a cross sectional view taken along the line in FIG. 2.

FIG. 4 is an enlarged view showing a bottom section, a fuel introducing part, and a lower fuel discharging part of the sub-tank illustrated in FIG. 2.

FIG. 5 is an enlarged view showing an upper section and an upper fuel discharging part of the sub-tank illustrated in FIG. 2.

### EMBODIMENT OF THE INVENTION

An embodiment of the present invention is described below with reference to the drawings.

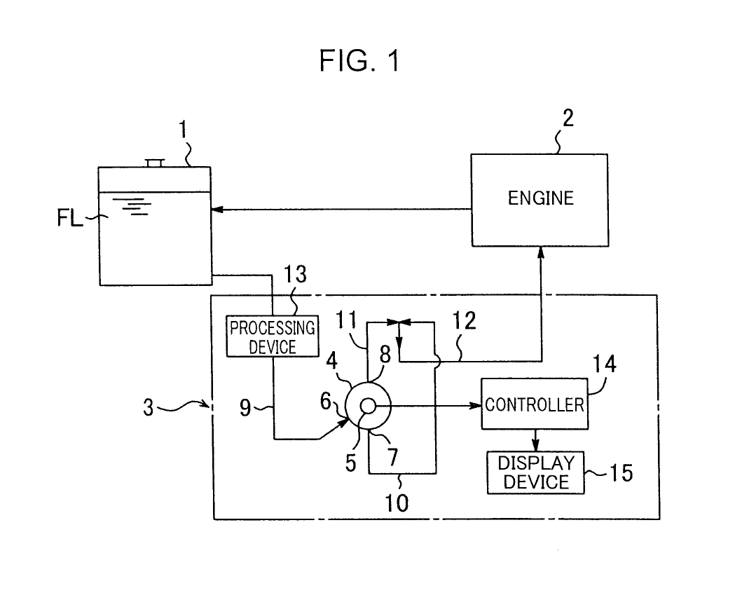

FIG. 1 illustrates a fuel property detection apparatus according to the embodiment. The apparatus is provided in a construction machine, as illustrated in FIG. 1, including an engine 2 and a fuel tank 1 for containing fuel FL to be used for the engine 2. The apparatus constitutes at least a part of a fuel supply line 3 for supplying the fuel FL from the fuel tank 1 to the engine 2, and performs detection of a property of the supplied fuel FL. The apparatus includes a sub-tank 4, a sensor 5, a fuel introducing pipe 9, and a fuel discharger including a bottom fuel discharging pipe 10, an upper fuel discharging pipe 11, and a junction pipe 12.

The sub-tank 4 is disposed between the engine 2 and the fuel tank 1 to contain the fuel FL to be supplied to the engine 2 from the fuel tank 1. The sub-tank 4 is a small-sized tank having a capacity smaller than that of the fuel tank 1.

The sensor 5 is provided in the sub-tank 4 to detect a property of the fuel FL inside the sub-tank 4. Specifically, the sensor generates a detection signal in the form of an electric signal indicative of a property of the fuel FL. The property to be detected may be a physical quantity such as kinematic density or density, or may be a chemical property.

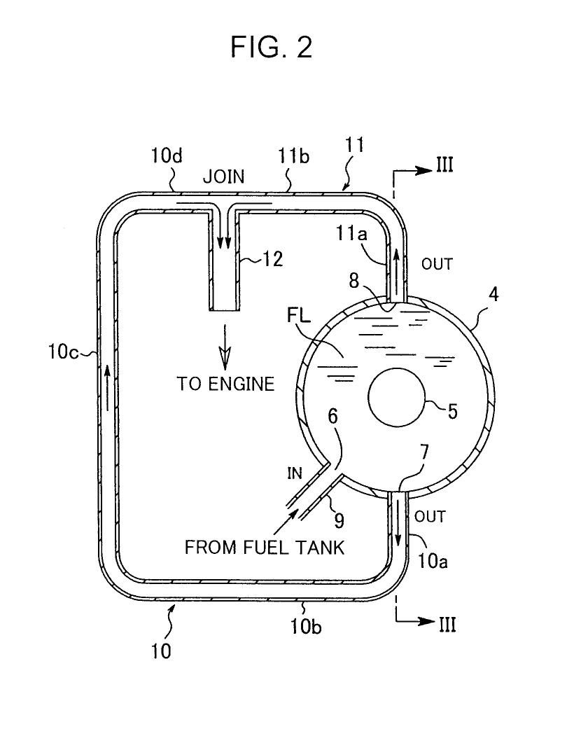

As also illustrated in FIG. 2 to FIG. 5, the sub-tank 4 according to the embodiment has a fuel inlet 6, a bottom fuel outlet 7, and an upper fuel outlet 8. Each of these is an opening which allows the fuel FL to pass through. The bottom fuel outlet 7 is formed in the bottom section of the sub-tank 4, and the upper fuel outlet 8 is formed in a top section of the sub-tank 4. In this embodiment, the fuel inlet 6 is formed at a location which is in a lower section of the sub-tank 4 but slightly upper of the bottom fuel outlet 7.

The fuel introducing pipe 9 is provided between the fuel tank 1 and the sub-tank 4 to allow the fuel FL to be supplied from the fuel tank 1 to the sub-tank 4 through the fuel introducing pipe 9. The fuel introducing pipe 9 has a downstream end connected to the fuel inlet 6, through which the fuel FL is introduced into the sub-tank 4. The bottom fuel discharging pipe 10 has an upstream end connected to the bottom fuel outlet 7, and the upper fuel discharging pipe 11 has an upstream end connected to the upper fuel outlet 8. Accordingly, the fuel FL in the sub-tank 4 can be discharged to the engine 2 from the bottom section of the sub-tank 4 via the bottom fuel outlet 7 and the bottom fuel discharging pipe 10, and can also be discharged to the engine 2 from the upper section of the sub-tank 4 via the upper fuel outlet 8 and the upper fuel discharging pipe 11.

The bottom fuel discharging pipe 10 and the upper fuel discharging pipe 11 have respective downstream ends which are connected to a common junction pipe 12 so as to join to each other at the junction pipe 12. The junction pipe 12 has an upstream end connected to the fuel discharging pipes 10 and 11, and a downstream end connected to the engine 2.

In the embodiment, as shown in FIG. 1, a processing device 13 is provided in an intermediate location of the fuel introducing pipe 9. The processing device 13 comprises a water separator, a filter, and like members. Alternatively, the processing device 13 may be provided in the junction pipe 12.

Furthermore, the fuel supply line 3 according to the embodiment includes a fuel pump (not illustrated). The fuel pump has a pressurizing function for causing the fuel FL in the fuel tank 1 to flow to the engine 2 through the fuel supply line 3.

The sub-tank 4, provided in the fuel supply line 3 as described above, is able to decrease the flow velocity of the fuel FL in the sub-tank 4 and stabilize flow rate thereof. The sensor 5, thus generating a detection signal regarding a property of the fuel FL in the sub-tank 4 under such a condition that the fuel FL has the decreased flow velocity and the stabilized flow rate, brings about the basic effect of securing the time required for detecting a fuel property and the stabilized flow rate.

The sub-tank 4 can be favorably mounted on a suitable part of the construction machine, for example on an upper frame of an upper slewing body in the case of a hydraulic excavator. That is because: (I) as the position on which the sub-tank 4 is to be provided, any advantageous position in terms of space and mountability can be selected among positions between the fuel tank 1 and the engine 2, and (II) the sub-tank 4 can be a small-sized tank because it only has to have a capacity enough to decrease the flow velocity to one required for detecting a property of the fuel FL. This apparatus thus allows the accuracy and the stability in the detection of a fuel property to be basically improved while employing the sub-tank type capable of being additionally installed onto an existing machine and having good mountability.

In the example illustrated in FIG. 1, the detection signal generated by the sensor 5 is inputted into a controller 14. The controller 14 determines adequacy of the fuel FL based on the detection signal, and causes a display device 15 to perform display, warning, and the like.

Next described in details are configurations of the sub-tank 4 and its related parts.

In the embodiment, the sub-tank 4 has a cylindrical peripheral wall having a horizontal central axis. The fuel inlet 6 is formed in a substantially center location in a longitudinal direction, i.e., the axis direction, of the peripheral wall, and is obliquely downwardly directed toward the outside of the sub-tank 4 in axial view, as illustrated in FIG. 2. The bottom fuel outlet 7 is vertically downwardly directed toward the outside of the sub-tank 4, and the upper fuel outlet 8 is vertically upwardly directed toward the outside of the sub-tank 4.

As illustrated in FIG. 2 and other drawings, the bottom fuel discharging pipe 10 according to the embodiment has an upstream vertical section 10a, an intermediate horizontal section 10b, a downstream vertical section 10c and a downstream horizontal section 10d. The upstream vertical section 10a extends vertically, and has an upper end connected to the bottom fuel outlet 7. The intermediate horizontal section 10b extends horizontally from a lower end of the upstream vertical section 10a. The downstream vertical section 10c extends vertically upwardly from one end of opposite ends of the intermediate horizontal section 10b, the one end being opposite to the intermediate horizontal section 10b, the other end connected to the upstream vertical section 10a. The downstream horizontal section 10d extends horizontally in such a direction as to come closer to the sub-tank 4 from an upper end of the downstream vertical section 10c.

The upper fuel discharging pipe 11 has a vertical section 11a and a horizontal section 11b. The vertical section 11a extends vertically and has a lower end connected to the upper fuel outlet 8. The horizontal section 11b extends horizontally in such a direction as to come closer to the downstream horizontal section 10d of the bottom fuel discharging pipe 10 from an upper end of the vertical section 11a.

The downstream horizontal section 10d and the horizontal section 11b have respective downstream ends which are connected to an upstream end of the junction pipe 12 so as to join to each other at the common junction pipe 12. This connection enables the fuel FL flowing in the upper fuel discharging pipe 11 to join to the fuel FL discharged through the bottom fuel discharging pipe 10 at the highest part of the upper fuel discharging pipe 11.

The fuel introducing pipe 9 and the fuel discharging pipes 10 and 11 are constituted by, for example, an inflow connector steel cylinder and outflow connecting steel cylinders connected to the sub-tank 4, and hoses connected to the steel cylinders, respectively. Meanwhile, in FIG. 2, each of the pipes 9 to 11 is indicated just as single pipe for simplification. Similarly, the junction pipe 12, which is constituted by, for example, a T-shaped joint provided in the joining part and a hose connected to the joint, is indicated as one pipe in FIG. 2.

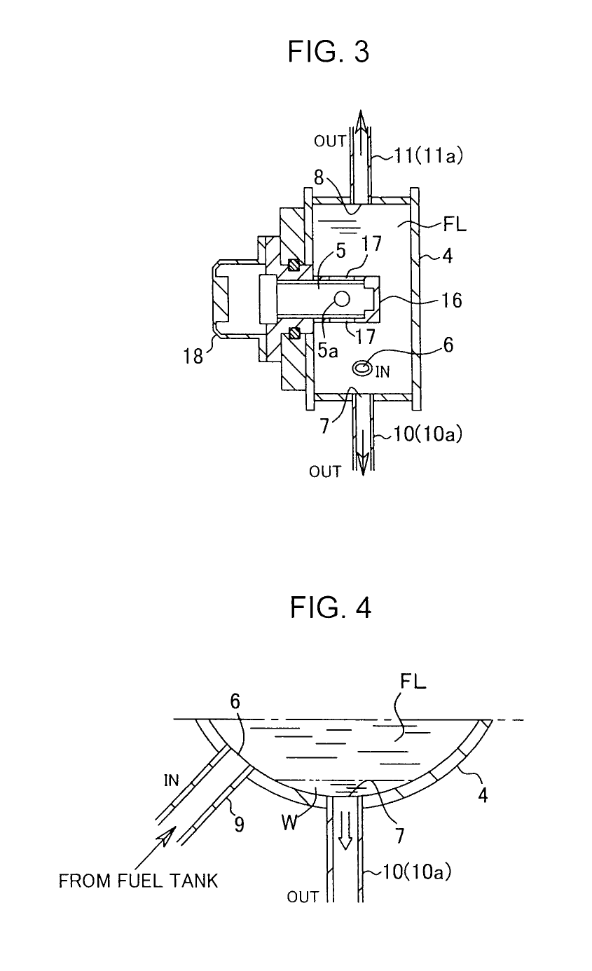

FIG. 3 shows an example of a configuration for attachment of the sensor 5. The sensor 5 has a sensing section 5a and is attached to one of the longitudinally opposite walls of the sub-tank 4 so as to allow the sensing section 5a to make contact with the fuel FL in the sub-tank 4 at a middle position (a center or a position near to the center) in the height direction of the sub-tank 4 and a middle position (a center or a position near to the center) in the longitudinal direction of the sub-tank 4.

The sensor 5 according to the embodiment is attached to the sub-tank 4 while being contained in a sensor case 16 as shown in FIG. 3. The sensor case 16 has upper and lower fuel passage holes 17 that bring the inside and outside of the sensor case 16 into communication with each other. The fuel passage holes 17 allows the fuel FL in the sub-tank 4 to enter inside the sensor case 16 through the fuel passage hole 17 to enable the sensor 5 to detect the property of the fuel FL. Besides, the sensor case 16 has a sensor cover 18, which covers an inner space of the sensor case 16 outside the sub-tank 4.

In the fuel property detection apparatus, the fuel FL in the fuel tank 1 is introduced into the sub-tank 4 through the fuel introducing pipe 9 and the fuel inlet 6. Only a part of the inflowing fuel FL is held in the sub-tank 4, and the other part exceeding the certain amount is discharged from the sub-tank 4 to the engine 2 through the bottom fuel outlet 7 and the fuel discharging pipe 10 and through the upper fuel outlet 8 and the fuel discharging pipe 11. The sensor 5 comes into contact with the fuel FL contained in the sub-tank 4 to generate a detection signal concerning the property of the fuel FL.

In the case where the fuel FL contains a bit of water, for example, the water having failed to be removed in the processing device 13, the bit of water, which has a greater specific gravity than the fuel FL, is liable to gradually separate from the fuel FL and accumulate on the bottom section of the sub-tank 4. In this situation, without the bottom fuel outlet 7 and the bottom fuel discharging pipe 10, the water could accumulate on the bottom section of the sub-tank as illustrated in FIG. 4 and the accumulated water might be moved in the sub-tank together with fuel by the flow of the fuel FL within the sub-tank or stirring currents accompanied by the flow of the fuel, thus coming into contact with a sensor to give bad effects to the detection of the sensor.

In contrast, in the above-described apparatus with the bottom fuel outlet 7 in the bottom section, that is, the lowest part, of the sub-tank 4, and the bottom fuel discharging pipe 10 connected to the bottom fuel outlet 7, the water is forcibly discharged to the downstream side through the bottom fuel discharging pipe 10 by the kinetic energy of the outflowing fuel FL through the lower fuel outlet 7, specifically, a flowing energy due to the pressurization by the fuel pump and a falling energy due to the self-weight of the fuel FL. Water is thus prevented from accumulating on the bottom section of the sub-tank 4. Even if having accumulated, the water is efficiently discharged from the sub-tank 4. This makes it possible to reduce the amount of water existing in the sub-tank 4 to minimize the influence of water on the detection of the sensor 5.

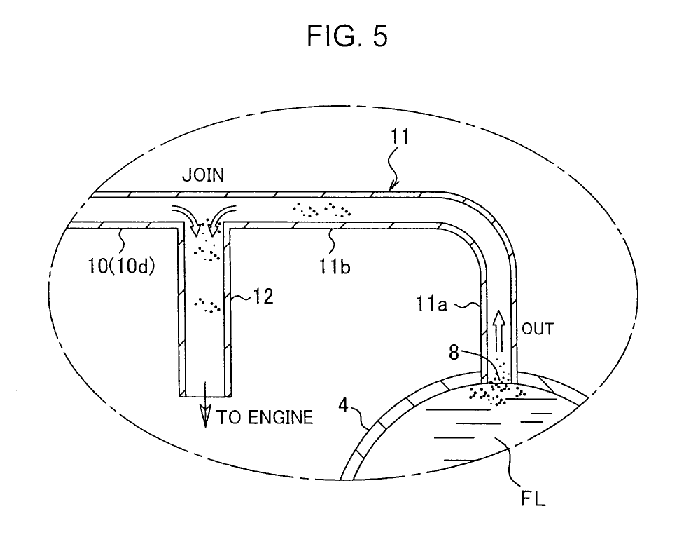

On the other hand, a bit of air contained in the fuel FL rises in the sub-tank 4, opposite to the water. If air accumulating on the top section of the sub-tank 4, the air would come into contact with the sensor 5 due to the flowing and stirring currents of the fuel FL to thereby cause bad effects to the property detection of the fuel FL. However, in the apparatus according to the embodiment, the sub-tank 4, having the upper fuel outlet 8 in its top section as well to which the upper fuel discharging pipe 11 is connected, allows the air accumulating on the top section of the sub-tank 4 to be flown out to the downstream side by the flowing energy of the fuel FL, as schematically shown by black dots in FIG. 5. In the embodiment, the bad influence of air to the detecting performance is thus minimized.

Furthermore, the discharged fuel FL flowing through the upper fuel discharging pipe 11 according to the embodiment joins to the discharged fuel FL flowing in the bottom fuel discharging pipe 10 at the highest part of the upper fuel discharging pipe 11 by the way of the junction pipe 12, thus enabling the air to flow into the junction pipe 12 owing to the joining energy. This prevents the air from accumulation in the upper fuel discharging pipe 11 to improve the flow of the fuel FL, thus allowing the air discharging performance to be enhanced.

Moreover, in this embodiment, the contact of the sensing section 5a of the sensor 5 with the fuel FL at a vertically middle section of the sub-tank 4, that is, at the section having less respective distributions of water and air than the bottom and top sections of the sub-tank 4, allows the detection accuracy to be enhanced.

For the above-described reasons, the fuel property detection apparatus can suppress the bad influences of water and air securely to thereby carry out detection of a fuel property in a high accuracy.

The present invention is not limited to the embodiment described above, including, for example, the following embodiments.

(1) The fuel inlet 6 of the sub-tank 4, which is formed at the obliquely lower left portion as shown in FIG. 2, is also permitted to be formed at a position laterally symmetrical to the obliquely lower left position, that is, at an obliquely lower right position, at a position vertically symmetrical to the obliquely lower left position or the obliquely lower right position, that is, at an obliquely upper left position or an obliquely upper right position, or at either a left or right position in a middle part of the vertical length of the sub-tank 4. Alternatively, a plurality of fuel inlets 6 may be formed at respective plural positions.

(2) The shape of a sub-tank is not limited to that shown in the above embodiment, according to the present invention. In the viewpoint of facilitating the discharge of water and air owing to easy restriction of an area for collecting them, it is preferable to provide a sub-tank having a cylindrical shape extending horizontally as the sub-tank 4 illustrated in FIG. 2 and other drawings; a sub-tank according to the present invention, however, may have a cylindrical shape with a vertical axis or a rectangular-parallelepiped shape.

(3) The position of the sensor is not limited to that shown in the above embodiment, according the present invention. The sensor according to the present invention may be provided at a position lower or upper than the middle of the vertical length of the sub-tank, or at a position offset longitudinally of the sub-tank.

As described above, the present invention provides an apparatus which is used for a construction machine including an engine and a fuel tank to detect a property of fuel and capable of detecting a property of fuel securely and with high accuracy with use of a sub-tank. Specifically, the apparatus is to be provided on a construction machine including an engine and a fuel tank for containing fuel for supply to the engine to detect a property of fuel, the apparatus comprising: a sub-tank provided between the engine and the fuel tank to contain fuel to be supplied from the fuel tank to the engine; a fuel introducing pipe provided between the sub-tank and the fuel tank to allow fuel to be introduced from the fuel tank to the sub-tank through the introducing pipe; a fuel discharger provided between the sub-tank and the engine to allow fuel to be discharged from the sub-tank to the engine through the discharger; and a sensor provided in the sub-tank to generate a detection signal concerning a property of the fuel in the sub-tank. The sub-tank has a bottom section having a bottom fuel outlet for discharging fuel from the bottom section. The fuel discharger includes a bottom fuel discharging pipe connected to the bottom fuel outlet.

In the apparatus, the water which has accumulated or tends to stay on the bottom section of the sub-tank is forcibly discharged to the downstream side by a kinetic energy possessed by the outflowing fuel from the bottom section of the sub-tank, specifically, a flowing energy caused by the applied pressure by a fuel pump and a falling energy caused by the self-weight of the fuel, thereby accomplishing efficient discharge of water from the sub-tank: this makes it possible to effectively suppress the influences of the water in the sub-tank to the detection.

It is preferable that the sub-tank further has an upper fuel outlet positioned in a top section of the sub-tank, and the fuel discharger further includes an upper fuel discharging pipe connected to the upper fuel outlet. The air contained in the fuel is likely to accumulate on a highest part of the sub-tank, opposite to water, and, if accumulating, the air could be moved in the fuel by the flowing and stirring currents of the fuel to come into contact with the sensor, thus causing bad influences to the fuel property detection, similarly to water; however, the upper fuel outlet and the upper fuel discharging pipe connected to the upper fuel outlet allows the accumulated air on the highest part of the sub-tank to be flown out to the downstream side due to the flowing energy of the fuel to be discharged from the sub-tank, thus also making it possible to suppress the bad influences of air to the detection performance.

In this arrangement, it is preferable that the fuel discharger further includes a junction pipe connected to the bottom fuel discharging pipe and the upper fuel discharging pipe to allow the fuel flowing through the bottom fuel discharging pipe to join to the fuel flowing through the upper fuel discharging pipe at the junction pipe and flow to the engine, the upper fuel discharging pipe being connected to the junction pipe at a highest part of the upper fuel discharging pipe. The respective fuels flowing through the bottom fuel discharging pipe and through the upper fuel discharging pipe can join to each other at the highest part of the upper fuel discharging pipe, thereby enabling the air to be flown into the junction pipe by the joining energy. This can prevent the air from accumulating in the upper fuel discharging pipe, and improve the flow of fuel, thus allowing the air discharging performance and further detection accuracy to be enhanced.

It is preferable that the sensor includes a sensing section and is provided at a vertically middle part of the sub-tank so as to allow the sensing section to make contact with the fuel in the sub-tank. This contact of the sensing section with the fuel at the vertically middle part of the sub-tank, where the distribution of water and air is less than the bottom and top sections of the sub-tank, enables the detection accuracy to be increased.

This application is based on Japanese Patent application No. 2013-175419 filed in Japan Patent Office on Aug. 27, 2013, the contents of which are hereby incorporated by reference.

Although the present invention has been fully described by way of example with reference to the accompanying drawings, it is to be understood that various changes and modifications will be apparent to those skilled in the art. Therefore, unless otherwise such changes and modifications depart from the scope of the present invention hereinafter defined, they should be construed as being included therein.

What is claimed is:

1. An apparatus to be provided on a construction machine including an engine and a fuel tank for containing fuel to be supplied to the engine, to detect a property of fuel, the apparatus comprising:
a sub-tank provided between the engine and the fuel tank to contain fuel to be supplied from the fuel tank to the engine;
a fuel introducing pipe provided between the sub-tank and the fuel tank to allow fuel to be introduced from the fuel tank to the sub-tank through the fuel introducing pipe;
a fuel discharger provided between the sub-tank and the engine to allow fuel to be discharged from the sub-tank to the engine through the fuel discharger; and
a sensor provided in the sub-tank to generate a detection signal concerning a property of the fuel in the sub-tank,
wherein the sub-tank has a bottom section having a bottom fuel outlet for discharging fuel from the bottom section, and the fuel discharger includes a bottom fuel discharging pipe connected to the bottom fuel outlet.

2. The fuel property detection apparatus for a construction machine as set forth in claim 1, wherein the sub-tank further has an upper fuel outlet in a top section of the sub-tank, and the fuel discharger further includes an upper fuel discharging pipe connected to the upper fuel outlet.

3. The fuel property detection apparatus for a construction machine as set forth in claim 2, wherein the fuel discharger further includes a junction pipe connected to the bottom fuel discharging pipe and the upper fuel discharging pipe to allow the fuel flowing in the bottom fuel discharging pipe to join to the fuel flowing in the upper fuel discharging pipe and flow to the engine, the upper fuel discharging pipe being connected to the junction pipe at a highest part of the upper fuel discharging pipe.

4. The fuel property detection apparatus for a construction machine as set forth in claim 1, wherein the sensor includes a sensing section and is provided in the sub-tank so as to allow the sensing section to make contact with the fuel in the sub-tank at a vertically middle part of the sub-tank.
